(19) United States
(12) Patent Application Publication
(10) Pub. No.: US 2006/0282111 A1
(43) Pub. Date: Dec. 14, 2006

(54) Segmented Embolectomy Catheter

(75) Inventor: Hesham Morsi, Houston, TX (US)

Correspondence Address: CONLEY ROSE, P.C., P. O. BOX 3267, HOUSTON, TX, 77253-3267, US

(73) Assignee: BAYLOR COLLEGE OF MEDICINE, Houston, TX (US)

(21) Appl. No.: 11/423,343

(22) Filed: Jun. 9, 2006

Related U.S. Application Data
(60) Provisional application No. 60/688,824, filed on Jun. 9, 2005.

(51) Int. Cl. A61M 29/00

(52) U.S. Cl. 606/194

(57) ABSTRACT

A discrete segmented balloon catheter for removal of an embolus. In an embodiment, the catheter comprises a catheter having a proximal and a distal end, a fluid inlet at the proximal end, and discrete expandable segments placed along the shaft of the catheter. In addition, the segments are expanded through dispersal of fluid that flows from the catheter's proximal inlet. It follows that the segments are contracted through withdrawal of fluid from the catheter's proximal inlet.

### CROSS-REFERENCE TO RELATED APPLICATIONS

This application claims benefit of U S. Provisional Application Ser. No. 60/688,824, filed on Jun. 9, 2005, which is incorporated herein by reference in its entirety.

### STATEMENT REGARDING FEDERALLY SPONSORED RESEARCH OR DEVELOPMENT

Not applicable

### FIELD OF THE INVENTION

The present invention relates generally to catheters and more particularly, to catheters for use as embolectomy catheters and angioplasty catheters for treating diseases including but not limited to stroke Still more particularly, the present invention relates to the use of balloon or expanding catheters for the treatment and/or removal of emboli.

### BACKGROUND

Expanding catheters are commonly used in surgical procedures to remove emboli or blood clots from an occluded branch or vessel. An embolus is most frequently a blood clot, but it can also be a plaque broken off from an atherosclerotic blood vessel or a number of other substances including fat, air, and even cancerous cells Typically, the catheter is inserted percutaneously to the vicinity of the clot and expanded, capturing a portion of the clot, which is then withdrawn from the vessel upon removal of the catheter. One mechanism for expansion of a catheter is inflation,

Catheters with inflatable balloon means have been provided for blood clot removal. U.S. Pat. No. 4,762,130 to Fogarty discloses such a catheter. The Fogarty device uses a single, spiral-configured balloon. U.S. Pat. No. 6,254,571 to Hart discloses a second type of catheter for removing occlusive materials from body passages, in which a plurality of mechanically activated expandable segments are disposed on the distal end of a catheter.

Embolectomy catheters have also been provided with balloons having small flexible protrusions adapted to bite into the clot upon inflation of the balloons, enabling a portion of the clot to be pulled free by withdrawal of the catheter. Such a catheter is shown in U.S. Pat. No, 3,635,223 to Klieman. Various other means for removing emboli exist, include coil-shaped and basket-shaped devices, which typically constructed of wire or the like. None of these are consistently effective for clot removal, largely because new clots tend to be less organized and therefore more delicate.

Existing expandable catheters may suffer from several other problems. For example, if such a catheter comprises a single balloon and it is inflated near a well-organized clot, expansion of the balloon may result in the application of excessive force to the delicate vessel wall. Second, if a single expanded balloon catches the clot and the clot is large, the process of removing the clot may also create excessive forces on the vessel. Such procedures may damage the wall of the vessel. In addition, expanding catheters that do not engage most of the clot mass may not trap and retain a large portion of the clot, especially upon withdrawal of the catheter from the vessel.

Accordingly, there remains a need in the art for an angioplasty or embolectomy catheter that can capture, retain, and remove all or a significant portion of the blood clot without producing excessive pressure on the vessel.

### SUMMARY OF SOME OF THE PREFERRED EMBODIMENTS

These and other needs in the art are addressed in one embodiment by a discrete segmented balloon catheter. In an embodiment, the present embolectomy catheter comprises a first catheter having a proximal and a distal end, a fluid inlet at the proximal end, and discrete expandable segments placed along the shaft of the catheter. In addition, the segments are expanded through dispersal of fluid that flows from the catheter's proximal inlet. The segments can be contracted through withdrawal of fluid from the catheter's proximal inlet.

An additional embodiment includes a guiding catheter in combination with the aforementioned first catheter, where a portion of the first catheter is inside the guiding catheter. Upon contraction or partial contraction of the expandable segments, some or all of the first catheter may be drawn into the guiding catheter, so as to allow at least a portion of the clot mass to be drawn into the guiding catheters.

At least a portion of an embolus may be removed by deploying the first catheter, with its segments contracted, along a guide wire into the embolus. Once positioned in the embolus, the segments may be expanded to capture a substantial amount of the embolus between the segments The first catheter is then withdrawn, removing the expanded segments and the embolus trapped between them from the occluded branch.

Thus, embodiments described herein comprise a combination of features and advantages intended to address various shortcomings associated with certain prior devices. The various characteristics described above, as well as other features, will be readily apparent to those skilled in the art upon reading the following detailed description of the preferred embodiments, and by referring to the accompanying drawings. It should be appreciated by those skilled in the art that the conception and the specific embodiments disclosed may be readily utilized as a basis for modifying or designing other structures for carrying out the same purposes of the embodiments described herein. It should also be realized by those skilled in the art that such equivalent constructions do not depart from the spirit and scope of the invention as set forth in the appended claims.

### BRIEF DESCRIPTION OF THE DRAWINGS

For a detailed description of the preferred embodiments of the invention, reference will now be made to the accompanying drawings in which:

FIG. 1 is a side cross-section through the longitudinal axis of a segmented balloon catheter constructed in accordance with a first embodiment the inventions

FIG. 2 is a cross-section nodal to the tool axis, taken through an expanded member of the catheter of FIG. 1

FIG. 3 illustrates three sequential views of the process for removing an embolus using the catheter.

FIG. 4 is a side cross-section through the longitudinal axis of a second embodiment of a segmented balloon catheter

### DETAILED DESCRIPTION OF THE PREFERRED EMBODIMENTS

The following discussion is directed to various embodiments of the invention. Although one or mote of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Certain terms are used throughout the following description and claims to refer to particular features or components. As one skilled in the art will appreciate, different persons may refer to the same feature or component by different names. This document does not intend to distinguish between components or features that differ in name but not function The drawing figures are not necessarily to scale. Certain features and components herein may be shown exaggerated in scale or in somewhat schematic form and some details of conventional elements may not be shown in interest of clarity and conciseness.

In the following discussion and in the claims, the terms “including” and “comprising” are used in an open-ended fashion, and thus should be interpreted to mean “including, but not limited to. . . ” Also, the term “distal” is intended to refer to positions relatively away from the operator of the catheter when it is in use, while the term “proximal” is intended to refer to positions relatively near the operator when the catheter is in use. As a result, the distal end of a device is relatively near the embolus as compared to the proximal end of the device, which is relatively away from the embolus In addition, the term “radial” is intended to refer to movement toward or away from the longitudinal central axis of the catheter The term “axial” is meant to refer to positions lengthwise along the central axis of the catheter. The term “discrete” is intended to describe members that are individually disposed and separately inflatable. If one discrete member is obstructed and unable to fully expand, the next discrete member is not affected and may be expanded to its predetermined shape without regard to other members.

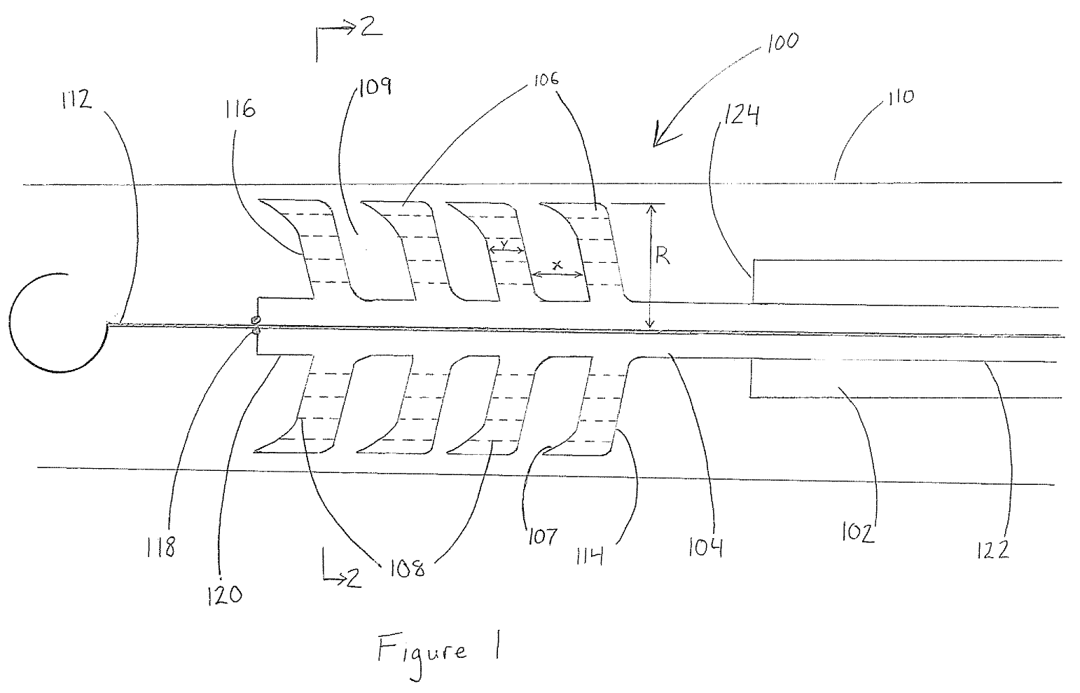

Referring initially to FIG. 1, according to a preferred embodiment, a catheter 100 is disposed inside a vessel 110 In preferred embodiments, catheter 100 includes a guiding catheter 102 and an inner catheter 104. A portion of inner catheter 104 may be disposed inside guiding catheter 102. The outer diameter of guiding catheter 102 is preferably less than the inner diameter of the occluded portion of the vessel and the outer diameter of inner catheter 104 is less than the inner diameter of guiding catheter 102.

By way of example only, in most adults, the common carotid artery has a diameter of about 6-10 mm, the internal carotid artery has a diameter of about 5-6 mill, and the middle cerebral artery has a diameter of about 2-3 mm. In embodiments of the present invention, the guiding catheter may be positioned upstream of the embolus, in the common or internal carotid artery, which has a diameter of 5-6 mm, so the guiding catheter may have a diameter of 2-3 mm. In these embodiments, the inner microcatheter 104 may have a diameter of 0.5 to 1 mm, so that it can enter the smaller vessel or branch where the embolus is located.

In a preferred embodiment, inner catheter 104 includes a plurality of separate or discrete annular expandable members 106 that are disposed axially along the shaft of the distal end 120 of inner catheter 104. The expanded outer diameter, R, of expandable members 106, is preferably approximately equal to vessel 110's inner diameter. Thus, in some embodiments, R will equal approximately 25 to 5 mm.

Inner catheter 104 and members 106 are in fluid communication with each other and expandable members 106 are expanded by filling inner catheter 104 and members 106 with fluid. In a preferred embodiment, the fluid disposed in inner catheter 104 may be comprised of radiopaque fluid, such as is well known in the art. As fluid travels from proximal end 122 toward distal end 120 of inner catheter 104, members 106 expand.

Members 106, according to a preferred embodiment, are constructed of a resilient material that allows for expansion and contraction, such as are known in the art. In an embodiment the resilient material comprises a polymer. In other embodiments, members 106 comprise a flexible material that does not appreciably stretch. Members 106 each include an inner wall 116 and an outer wall 114 Members 106 are preferably spaced axially along the shaft of catheter with a distance X between the inner wall I 16 of one member 106 and the outer wall 114 of an adjacent member 106 and a distance Y between the inner wall 116 and outer wall 114 of a given member 106. In some embodiments, X may be in the range of 1 to 5 mm and Y is in the range of 0.5 to 4 mm.

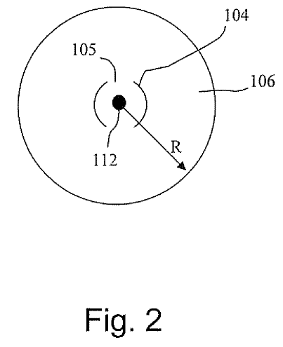

Referring now to FIG. 2, in certain embodiments, fluid enters each expandable member 106 from catheter 104 through at least one port 105 According to a preferred embodiment, the shaft of catheter 104 includes two opposing ports 105 for each member 106, as shown in FIG. 2. Ports 105 may be evenly spaced around the circumference of catheter shaft 104. Alternatively, ports 105 may be unevenly spaced or there may be only one port 105 for each expandable member 106. The axial extent of ports 105 may be equal to Y, or may be to 0.25 Y or less.

In a preferred embodiment, members 106, when expanded, form a predetermined shape such as circular, conical, or cup-shaped. Inner wall 116 forms the inner or distal surface of the cone or cup shape and outer wall 114 forms the outer surface of the shape. In the preferred expanded cup-shape, inner wall 116 will form an acute angle with the shaft of catheter 104 and outer wall 114 will form an obtuse angle with the shaft of catheter 104. While the figures illustrate a preferred embodiment, it will be understood that expandable members 106 may have other shapes, may be concave in the opposite direction, and/or may not all be identically sized or shaped.

Contraction of members 106 reduces the overall diameter of inner catheter 104, so that it is less than the inner diameter of guiding catheter 102. When members 106 are contracted, inner surface 116 is drawn toward the central axis of inner catheter 104.

In preferred embodiments, catheter 100 includes a radiopaque mark (not shown) on at least the most distal segment and the most proximal segment. Such radiopaque marks aid in visualization during placement and extraction, as described below.

In some embodiments, one or more webs 108 are disposed inside members 106. In preferred embodiments, webbing 108 is composed of a material that is less stretchy than members 106. Webbing 108 may be disposed in one or a plurality of places inside each member 106 and shaped such that it prevents deformation of members 106 beyond their predetermined shape. In the embodiment shown, multiple webs 108 connect each inner wall 116 to the outer wall 114 of the same expandable member 106, thereby limiting the ability of the member 106 to expand beyond the desired shape and more specifically limiting the ability of each member 106 to deform such that the distance between its inner and outer walls 116, 114 exceeds the desired distance, Y.

In preferred embodiments, each expandable member 106 is preferably concave when viewed from the distal end of the tool. Thus, as in the embodiment illustrated in FIG. 1, the outer edge 107 of each member 106 is closer to the distal end of the tool than is the central portion of each member. This concavity, coupled with the axial spacing of the members along the tool results in a capture space 109 that is defined between each adjacent pair of members 106. During an embolectomy, described below, portions of the embolus are captured in spaces 109. As the tool is retracted, the distally curving outer edges 107 help retain the captured portions.

A guide wire 112, such as is well known in the art, extends through the distal end of inner catheter 104 at seal 118. Seal 118 allows inner catheter 104 to travel along guide wire 112 for proper placement within vessel 110 as described below and prevents the egress of fluid around the guide wire during inflation of members 106.

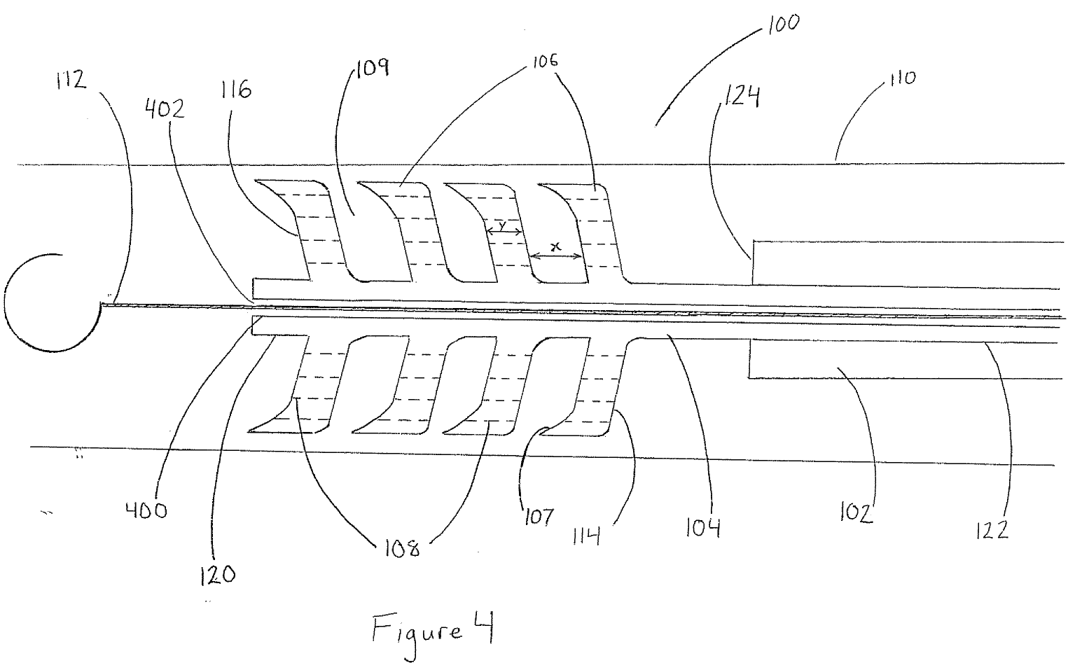

In an alternative embodiment, as shown in FIG. 4, inner shaft 400 is disposed inside inner catheter 104, forming inner lumen 402. Guide wire 112 extends through inner lumen 402. The distal end of inner shaft 400 is sealed with the shaft of inner catheter 104, so as to maintain the sealed fluid chamber defined by members 106 and inner catheter 104. Inner shaft 400 can be made of the same resilient or flexible material as inner catheter 104.

Catheter 100, including members 106, and guide wire 112 preferably comprise materials that are biocompatible and non-thrombogenic.

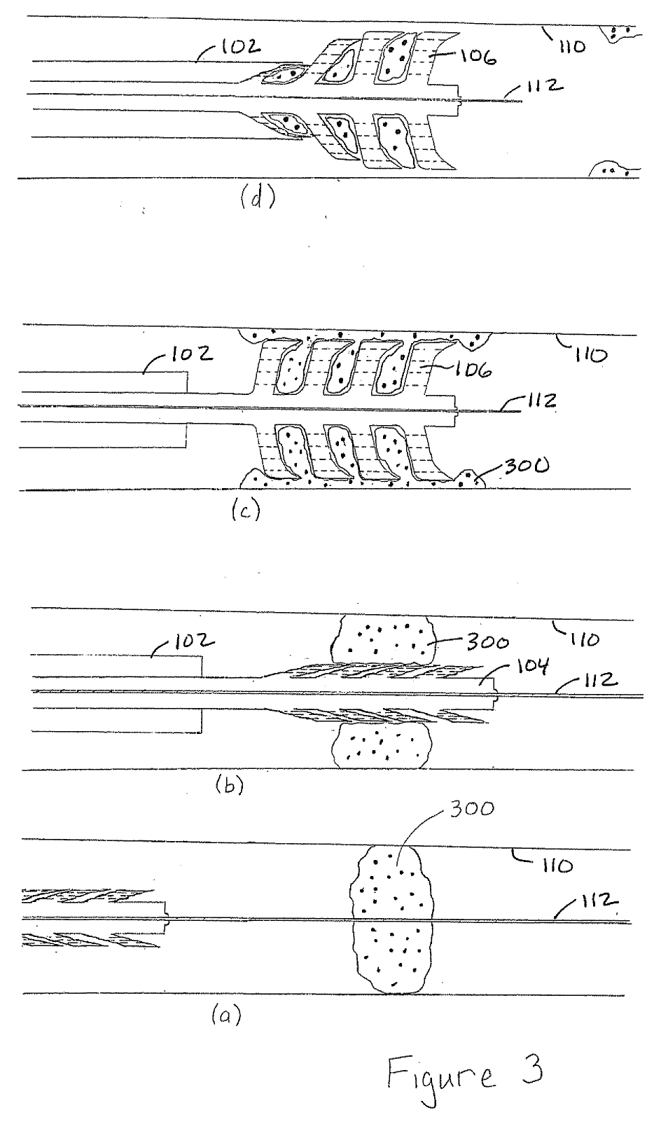

As illustrated in FIGS. 3(a)-(d), catheter 100 may be disposed in an occluded branch or vessel and used to remove an embolus 300 therefrom. To begin this operation, guide wire 112 is deployed in the vessel through and preferably somewhat beyond the embolus. Inner catheter 104 is then deployed so that its distal end 120 is disposed in the distal edge of the embolus 300, or otherwise as desired. During placement, members 106 are contracted and disposed adjacent to inner catheter 104 and guide wire 112. In a preferred embodiment, fluoroscopy or an equivalent technique is used to monitor the position of catheter 100 relative to embolus 300. In particular, radiopaque marks on the expandable segments can help ensure that the devices is positioned as desired.

Once in the desired position, ideally with members 106 fully embedded in embolus 300, members 106 are expanded as fluid flows from proximal end 122 to the distal end 120 of the device,. Members 106 gradually expand toward the inner wall of vessel 110, trapping portions of embolus 300 in spaces 109 between members 106.

After members 106 are expanded to a desired state and embolus 300 is captured between members 106, inner catheter 104 is drawn toward guiding catheter 102. As members 106 approach passage 124, fluid is gradually released from the distal end such that members 106 are contracted sequentially so as to maintain the trapping effect on a portion of the embolus while reducing R to allow members 106 to fit within guiding catheter 102 and reducing the overall volume of member 106, by also reducing Y, so as to allow capture of a maximum portion of the embolus. In some instances, collection of the embolus can be facilitated by applying suction to inside of guiding catheter 102.

Preferably after distal end 120 is drawn into guiding catheter 102, catheter 100 is withdrawn proximally from the occluded region, removing at least a portion of the embolus.

If embolus 300 is larger than can be removed by members 106 with one procedure, then the procedure may be repeated to remove the occlusion.

While a preferred embodiment of the invention is shown and described, it will be understood that variations to the embodiment can be made without departing from the scope of the present invention. Likewise, the sequential description or claiming of certain steps of the present method is not intended to limit the present method to performance of those steps in that order or in any particular order, unless otherwise stated.

What is claimed is:

1. A catheter comprising:
a catheter having a proximal end and a distal end;
a fluid inlet at said proximal end of said catheter; and
a plurality of discrete, annular expandable members disposed axially along said catheter in fluid communication with said inlet.

2. The catheter of claim 1, further comprising a guide wire disposed within said catheter.

3. The catheter of claim 2, further including a seal at said distal end of said catheter, wherein said guide wire extends through said seal.

4. The catheter of claim 2, further comprising an inner shaft defining an inner lumen disposed in said catheter, said guide wire being disposed in said inner lumen.

5. The catheter of claim 1 wherein said expandable members comprise a resilient material.

6. The catheter of claim 1 wherein said plurality of members are radially expandable.

7. The device of claim 1 wherein each of said plurality of members has an expanded outer diameter that is approximately equal to the vessel's inner diameter.

8. The catheter of claim 1 wherein said plurality of members are collapsible so as to reduce overall diameter of said catheter when contracted.

9. The catheter of claim 1 wherein each of said members, when expanded, has a circular, conical, or cup shape.

10. The catheter of claim 1 wherein said plurality of members have substantially identical expanded diameters.

11. The catheter of claim 1 wherein a web is affixed inside each of said plurality of members so as to prevent radial or axial deformation of said member beyond a predetermined extent.

12. The catheter of claim 11 wherein said web comprises a less resilient material than that of said plurality of members.

13. A device for removal of an embolus from a vessel comprising:
a guiding catheter having a first passage;
an inner catheter having a proximal end, a distal end, and an inlet at the proximal end of said inner catheter, wherein a portion of said proximal end is disposed within said first passage; and
a plurality of radially expandable members disposed axially along said inner catheter.

14. The device of claim 13 wherein each of said plurality of members has an expanded outer diameter that is approximately equal to the vessel's inner diameter.

15. The device of claim 11 further comprising a guide wire disposed within said catheter.

16. The device of claim 15, further including an inner shaft defining an inner lumen disposed in said catheter, said guide wire being disposed in said inner lumen.

17. The inner catheter of claim 13 wherein said plurality of members comprises a resilient material.

18. The catheter of claim 13 wherein a web is affixed inside each of said plurality of members so as to prevent radial or axial deformation of said member beyond a predetermined extent.

19. The catheter of claim 18 wherein said web comprises a less resilient material than that of said plurality of members.

20. A method of removing at least a portion of an embolus from a vessel comprising;
a) providing a first catheter comprising a plurality of radially expandable members disposed axially along said first catheter and a guide wire disposed within said catheter;
b) advancing said guide wire into the vessel distally through an occluded branch of the vessel;
c) inserting said first catheter along said guide wire into the occluded branch, wherein said plurality of members are contracted upon insertion;
d) expanding said plurality of members to capture at least a portion of an embolus between said members; and
e) withdrawing said first catheter and said expanded plurality of members proximally from said vessel to remove the embolus from the branch.

21. The method of claim 20 wherein (c) further comprises expanding said plurality of members to the occluded branch's diameter.

22. The method of claim 20 wherein (d) further comprises at least partially contracting said plurality of members upon proximally withdrawing said first catheter.

23. The method of claim 22 further comprising a guiding catheter, wherein a portion of said first catheter is disposed within said guiding catheter.

24. The method of claim 23 further comprising drawing said first catheter and said plurality of members into said guiding catheter and removing said guiding catheter and said first catheter from the vessel to extract at least a portion of the embolus.
